(19) United States
(12) Patent Application Publication
(10) Pub. No.: US 2025/0281132 A1
(43) Pub. Date: Sep. 11, 2025

(54) Integrated Detector Support for Mobile X-Ray Device

(71) Applicant: GE Precision Healthcare LLC, Waukesha, WI (US)

(72) Inventors: Beth A. Heckel, Waukesha, WI (US); Anna M. Keely, Waukesha, WI (US); Joseph W. Burge, Milwaukee, WI (US); Andrew G. Van Hulle, New Berlin, WI (US)

(21) Appl. No.: 18/596,004

(22) Filed: Mar. 5, 2024

(51) Int. Cl. A61B 6/00; A61B 6/10

(52) U.S. Cl. CPC A61B 6/4405; A61B 6/10; A61B 6/4452

(57) ABSTRACT

A mobile x-ray device includes a body including a number of wheels rotatably attached thereto and housing operative systems for the obtaining and processing of x-ray data to provide x-ray images, a moveable vertical column and horizontal arm operably connected to the body and including an x-ray emitter opposite the body, a detector configured to receive x-rays from the x-ray emitter and operably connected to the operative systems to transmit x-ray data from the detector to the operative systems, a drive handle disposed on the body, and a detector support having a sleeve defining an interior adapted to receive an edge of a detector therein, and a pair of securing plates extending outwardly from the sleeve and secured to the drive handle. The sleeve can securely engage opposed surfaces of the detector to allow the user/technician to readily position a protective bag around the exposed portion of the detector in a hands-free manner.

### FIELD OF THE DISCLOSURE

The present disclosure is related to x-ray imaging devices, and more particularly to detector management supports for mobile x-ray imaging devices.

### BACKGROUND OF THE DISCLOSURE

Digital imaging technologies including medical imaging technologies such as x-ray imaging allow for non-invasive acquisition of images of internal structures or features of a subject, such as a patient. Digital imaging systems produce digital data which can be reconstructed into radiographic images. In digital x-ray imaging systems, radiation from a source is directed toward the subject. A portion of the radiation passes through the subject and impacts a detector. The detector includes an array of discrete picture elements or detector pixels and generates output signals based upon the quantity or intensity of the radiation impacting each pixel region. The output signals are subsequently processed to generate an image that may be displayed for review. These images are used to identify and/or examine the internal structures and organs within a patient's body.

In order to facilitate obtaining x-ray images in a variety of locations and/or situation a number of mobile x-ray devices have been developed. These mobile x-ray devices have particular applicability in situations where the patient cannot or is not readily moveable, such that it is required to move the imaging device to the patient. The construction of the mobile x-ray device includes a mobile chassis that supports the entire x-ray assembly and a telescopic or fixed vertical column that is mounted on the chassis and can rotate about its long, vertical axis. The telescopic column includes a fixed portion jointed to the chassis, and one or more telescopic portions that are movably mounted to the fixed portion. The vertical column also includes a horizontal telescopic arm that can move along the column and that supports an x-ray emitter or source opposite the column.

In operation the mobile chassis can be moved into a location adjacent the patient or other object to be imaged. Then, employing the mechanisms disposed within the column and the telescopic arm, and the x-ray emitter can be moved to the desired position in order to obtain the x-ray images of the patient. The mechanisms allow the column and telescopic arm to be moved and counterbalanced by the remainder of the mobile x-ray device, thereby enabling the x-ray emitter to be properly and stably positioned to obtain the desired x-ray images.

The mobile x-ray device additionally includes one or more detectors associated with the mobile x-ray device. These detectors are capable of receiving the x-rays from the x-ray emitter that are directed through the patient and transmitting the information to the processing unit within the mobile chassis for generation of an x-ray image. The generated x-ray image can be presented on a display located on the mobile chassis in order to determine a proper course of treatment for the patient.

The detectors used in the imaging procedures performed with the mobile x-ray imaging device can be stored within a bin disposed on the chassis opposite the telescopic portion. The bin has dimensions that approximate the dimensions of the detectors, i.e., to enclose at least 50% to 67% of the area of the detector therein, such that the detectors can be securely retained within the bin during movement of the mobile x-ray device. The bin is disposed adjacent a bottom edge of the chassis to maintain the bin and detectors therein in an easy to access location that does not interfere with the operation and/or movement of the mobile x-ray device by the operator.

The detectors used with the mobile x-ray device can be operably connected by a wire or tether, or can be wirelessly connected to the processing device within the mobile chassis in order to transmit information and image data between the detector and the mobile chassis. To facilitate the handling of the detectors to place the detector in the desired position relative to the patient to obtain the desired image data to generate the x-ray image, the detector being used can be positioned within a grid handle or other removeable housing to provide a protective and more easily handled frame for the detector.

In use, as the detector is often placed in direct contact with the patient, to provide an amount of protection to the detector when in use, the detector and grid handle are placed within a protective bag or enclosure. The bag includes an open end through which the detector is inserted into the bag, and which is closed in a suitable manner when the detector is properly positioned within the bag.

The process for safely handling and inserting the detector or detector with grid/grid handle to place the detector within the bag prior to use can present several challenges. More specifically, due to the size of the detector, which can be up to 17″×17″ in size and weight up to 12 pounds, on many occasions the operator must balance the detector on a surface with one hand while placing the bag around the detector using the opposite hand. For example, the process of bagging the detector often involves placing the detector on the sloped top cover of the mobile x-ray device including the display, or on a near-by surface, such as a body part of the operator. In these scenarios, there is a significant potential for the detector to be placed in a position where it is not stable and to slide off of the surface leading to a detector drop event with the consequent negative effects leading to potential image quality issues or worse, detector failure. Further, the act of positioning and/or dropping the detector on the surface of the mobile x-ray device, i.e., on the display, can have negative consequences for the mobile x-ray device itself, e.g., damaging the display of the mobile x-ray device.

Additionally, the process of bagging the wireless or tethered detector is often a chaotic/acrobatic dance of holding the detector in one hand, bag in the other, balancing the detector on a body part, e.g., a knee for support of the detector and ultimately getting the bag onto the detector without dropping the detector. The operator may also rest the detector against their feet/shins to place the detector in the bag prior to use. The same scenarios outlined above often also occur when attempting to assemble a grid handle around a detector or remove the detector from it, or when removing the detector from within a bag after using in an imaging procedure. Each of these situations creates a poor ergonomic condition, and each situation can occur multiple times throughout the course of a single work shift, thereby creating the potential for injury to the operator.

Along with the issues created by the requirement for bagging the detector prior to each use in an imaging procedure, the mobile x-ray device includes an exposure switch that is connected to the chassis in order to enable the operator to selectively operate the x-ray emitter and obtain x-ray data from the detector in an imaging procedure. The exposure switch is connected to the chassis by an elongate cord that has a length sufficient to enable the operator to move a sufficient distance from the mobile x-ray device when the x-ray emitter is operated.

However, as a result of the length of the cord and/or the location of the connection of the cord to the chassis near the bin, the cord can often become positioned and tangled with the detectors in the bin requiring the operator to move the cord out of the way to position the detector and associated grid handle into or out of the bin. In addition, the exposure switch cord can also become entangled within a drive wheel of the mobile x-ray device or simply drag on the floor, neither of which is a desirable situation.

In order to alleviate these issues with the detectors, certain prior art mobile x-ray devices have incorporated notches within the chassis that accommodate the corners of the detector. These notches allow the corners of the detector to be inserted therein to function as the support for the detector on the chassis when a bag is positioned over or removed from the detector.

However, the location and sizes of the notches, either within a top surface of the chassis immediately adjacent the display or on the drive handle, only allow for the engagement with detectors of specific sizes, and only with or without grid handles attached. The notches can only securely engage the corners having a specified detector width, either with or without a grid handle, but not both. Also, the engagement of only the corners of the detector by the notches requires precise placement of both corners within the notches to effectively support the detector therein. In addition, the significantly limited areas of the detectors that can be engaged within the corners enable the corners to be easily disengaged from within the corners, particularly when the detector is inadvertently contacted, which can readily result in dropping the detector.

As a result, it is desirable to develop a detector support for a mobile x-ray device that has a simplified construction that enables effective and reliable engagement of detectors of various sizes therein that avoids the shortcomings of the prior art.

### SUMMARY OF THE DISCLOSURE

According to one aspect of an exemplary embodiment of the disclosure, a detector support for a mobile x-ray device includes a sleeve defining an interior adapted to receive an edge of a detector with or without a grid handle therein, and a pair of securing plates extending outwardly from the sleeve and adapted to be secured to the mobile x-ray device.

According to another exemplary embodiment of the disclosure, a method of supporting a detector on a mobile x-ray device includes the steps of providing a mobile x-ray device including a body including a number of wheels rotatably attached thereto and housing operative systems for the obtaining and processing of x-ray data to provide x-ray images, a moveable vertical column and horizontal arm operably connected to the body and including an x-ray emitter opposite the body, a detector configured to receive x-rays from the x-ray emitter and operably connected to the operative systems to transmit x-ray data from the detector to the operative systems, a drive handle disposed on the body, and a detector support having a sleeve defining an interior adapted to receive an edge of a detector therein, and a pair of securing plates extending outwardly from the sleeve and secured to the drive handle, placing an edge of the detector with or without a grid handle within the sleeve to support the detector within the sleeve in a hands-free manner, and placing a protective bag around the detector.

According to still another aspect of an exemplary embodiment of the present disclosure, a mobile x-ray device includes a body including a number of wheels rotatably attached thereto and housing operative systems for the obtaining and processing of x-ray data to provide x-ray images, a moveable vertical column and horizontal arm operably connected to the body and including an x-ray emitter opposite the body, a detector configured to receive x-rays from the x-ray emitter and operably connected to the operative systems to transmit x-ray data from the detector to the operative systems, a drive handle disposed on the body, and a detector support having a sleeve defining an interior adapted to receive an edge of a detector with or without a grid handle therein, and a pair of securing plates extending outwardly from the sleeve and secured to the drive handle.

These and other exemplary aspects, features and advantages of the invention will be made apparent from the following detailed description taken together with the drawing figures.

### BRIEF DESCRIPTION OF THE DRAWINGS

The drawings illustrate the best mode currently contemplated of practicing the present invention.

In the drawings:

FIG. 1 is a side elevation view of a mobile x-ray device, according to an exemplary embodiment of the disclosure.

FIG. 2 is a rear elevation view of the mobile x-ray device of FIG. 1 with a detector support disposed on the drive handle for the device, according to an exemplary embodiment of the disclosure.

FIG. 3 is a partially broken away, isometric view of the drive handle for the mobile x-ray device of FIG. 1 with the detector support attached thereto, according to an exemplary embodiment of the disclosure.

FIG. 4 is an isometric view of the detector support of FIG. 3, according to an exemplary embodiment of the disclosure.

FIG. 5 is a cross-sectional view along line 5-5 of FIG. 4.

FIG. 6 is an isometric view of the detector support of FIG. 3, according to another exemplary embodiment of the disclosure.

FIG. 7 is a cross-sectional view along line 7-7 of FIG. 6.

FIG. 8 is a partially broken away, isometric view of the detector support of FIG. 2 including a detector positioned therein, according to an exemplary embodiment of the disclosure.

FIG. 9 is a partially broken away, isometric view of the detector support of FIG. 2 including a detector positioned therein and in a bagged configuration, according to an exemplary embodiment of the disclosure.

FIG. 10 is a partially broken away, isometric view of the detector support of FIG. 2 including a detector positioned therein and being disengaged from a grid handle, according to an exemplary embodiment of the disclosure.

### DETAILED DESCRIPTION OF THE DRAWINGS

One or more specific embodiments will be described below. In an effort to provide a concise description of these embodiments, all features of an actual implementation may not be described in the specification. It should be appreciated that in the development of any such actual implementation, as in any engineering or design project, numerous implementation-specific decisions must be made to achieve the developers specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

When introducing elements of various embodiments of the present invention, the articles “a,” “an,” “the,” and “said” are intended to mean that there are one or more of the elements. The terms “comprising,” “including,” and “having” are intended to be inclusive and mean that there may be additional elements other than the listed elements. Furthermore, any numerical examples in the following discussion are intended to be non-limiting, and thus additional numerical values, ranges, and percentages are within the scope of the disclosed embodiments.

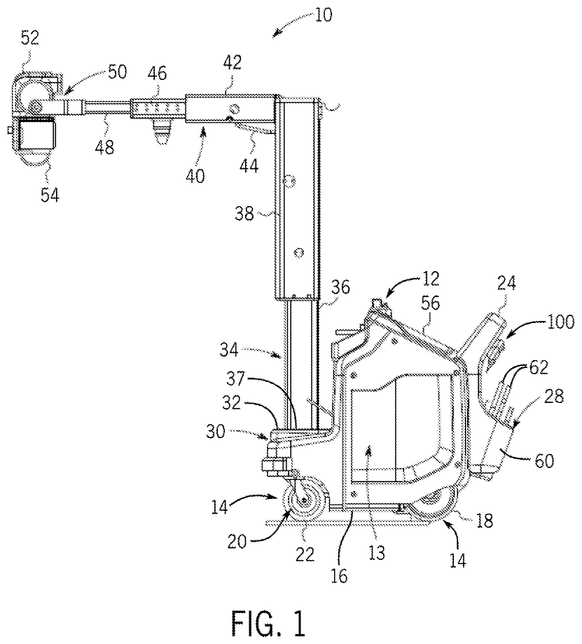

In FIG. 1, an exemplary embodiment is illustrated of a mobile x-ray device 10 constructed according to the disclosure. The mobile x-ray device 10 includes a chassis 12 that defines a body 13 including a number of wheels 14 attached to a lower surface 16 of the body 13 in order to allow the mobile x-ray device 10 to be moved over a surface by a technologist. The wheels 14 can include a pair of large support wheels 18 located on an axle (not shown) directly secured to the chassis 12, and one or more directional wheels 20 affixed to the chassis 12 in a pivotal manner, such as casters 22, to facilitate the movement of the chassis 12 in a desired direction. The chassis 12 can additionally include a drive handle 24 extending outwardly from the chassis 12 and graspable by a technician in order to manually direct the movement of the chassis 12 where desired. In an alternative embodiment or as an addition to the above embodiment, the chassis 12 may include a suitable motor (not shown) that is operably connected to the wheels 14, such as to the support wheels 18, in order to provide motorized movement capability to the chassis 12.

The body 13 of the chassis 12 defines a rear end 28 and a front end 30. Within the rear end 28 the body 13 encloses a number of operative computers, processing units and/or systems (not shown) as are well-known for use in the mobile x-ray device 10 such as the processing units and/or operative systems for the obtaining and processing of x-ray data to provide x-ray images using the mobile x-ray device 10. The front end 30 defines a platform 32 that supports that supports a vertical column 34 that can be moved as desired to position an x-ray emitter 52 attached to the column 34 where necessary to obtain the x-ray images.

The column 34 can optionally be telescopic and includes a lower fixed portion 36 disposed on the platform 32, where the fixed portion 36 is attached to the platform 32 by a suitable rotation mechanism (not shown) by a rotational collar 37 secured to the fixed portion 36 and rotatable with respect to the platform 32, that enables the fixed portion 36 to rotate with regard to the platform 32 along a vertical axis extending upwardly from the platform 32 through the fixed portion 36.

The column 34 additionally includes an upper telescopic portion 38 that is moveably attached to the fixed portion 36, such by a number of bearings (not shown) attached to the telescopic portion 38 and moveably disposed within guides (not shown) formed in the fixed portion 36. The upper telescopic portion 38 can move vertically with respect to the fixed portion 36.

Opposite the lower fixed portion 36, the upper telescopic portion 38 supports a horizontal telescoping arm 40. The telescoping arm 40 is movable vertically with regard to the telescopic portion 38. In an exemplary embodiment, the arm 40 is engaged with the telescopic portion 38 by a number of bearings (not shown) attached to the telescoping arm 40 and moveably disposed within guides (not shown) formed in the telescopic portion 38.

The telescoping arm 40 includes a fixed section 42 secured to a carriage 44 that is movably disposed on the telescopic portion 38, such as by bearings (not shown) formed on the carriage 44 and engaged in guides (not shown) in the telescopic portion 38, to provide the vertical movement of the telescoping arm 40 relative to the telescopic portion 38 of the column 34. A number of independently moveable sections 46,48 are secured to the fixed section 44 and can be selectively moved in a horizontal direction with regard to the fixed section 44 and one another to extend and retract the telescoping arm 40 relative to the telescopic portion 38.

Opposite the telescoping portion 38 the telescoping arm 40 supports a head assembly 50 on the outermost moveable section 48. In the illustrated exemplary embodiment of FIG. 1, the head assembly 50 for obtaining x-ray images. The head assembly 50 in the illustrated embodiment includes an x-ray source or emitter 52 and a collimator 54, and is secured to the moveable section 48 in any suitable manner that enables the head assembly 50 to pivot and/or rotate relative to the moveable section 48 in order to position the emitter 52 where necessary to obtain the desired x-ray images.

The various components of the telescopic column 32, i.e., the fixed portion 36 and the telescopic portion 38, and the telescoping arm 40, i.e., the fixed section 44 and the moveable sections 46,48, are each formed of a material that is sufficiently rigid to support the various components of the x-ray device 10 that are attached thereto, while also be able to be formed with a hollow interior to enable various wiring and other operational connections between body 13 and the emitter 52, such as through an aperture (not shown) in the platform 32 beneath the column 34, for the operation of the x-ray device 10 to be made completely within the interior of the x-ray device 10.

The mobile x-ray device 10 additionally includes a user console/display 56 on the body 13 in front of the drive handle 24 for the display of x-ray images obtained by the x-ray device 10 and/or for the operational control of the emitter 52 to obtain the x-ray images. In other embodiments, the console 56 can be disposed on an arm (not shown) that can be grasped by the user/technician to control the movement of the x-ray device 10. The user console 56 can be rotated with regard to the arm between 0° and 90° on the horizontal axis between use and storage positions, and additionally can rotate between −180° and +180° on the vertical axis.

The rear end 28 additionally includes a storage bin 60 extending outward from the rear end 28 below the drive handle 24. The storage bin 60 is configured to retain a number of detectors 62 therein, each of the detectors 62 operably connected, e.g. via a wired connection (not shown) or wirelessly, to the operative systems disposed within the body 13 in order to able to be utilized with the x-ray device 10 in an imaging procedure. The bin 60 includes spaces therein for detectors 62 of differing sizes in order to hold the detectors 62 in a manner that prevents damage to the detectors 62 from outside contact and contact with one another, while allowing the detectors 62 to be connected to a power source within the chassis 12 in the bin 60 and allowing easy access to the detectors 62 disposed within the bin 60.

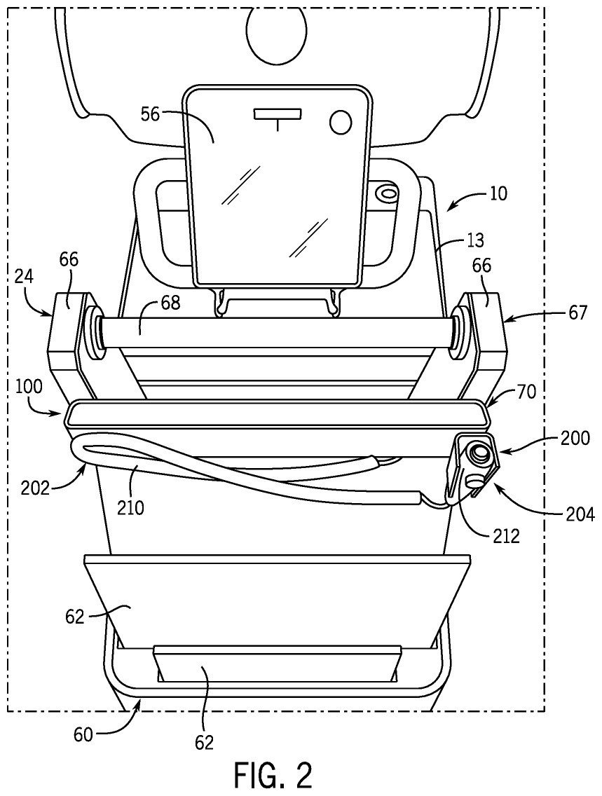

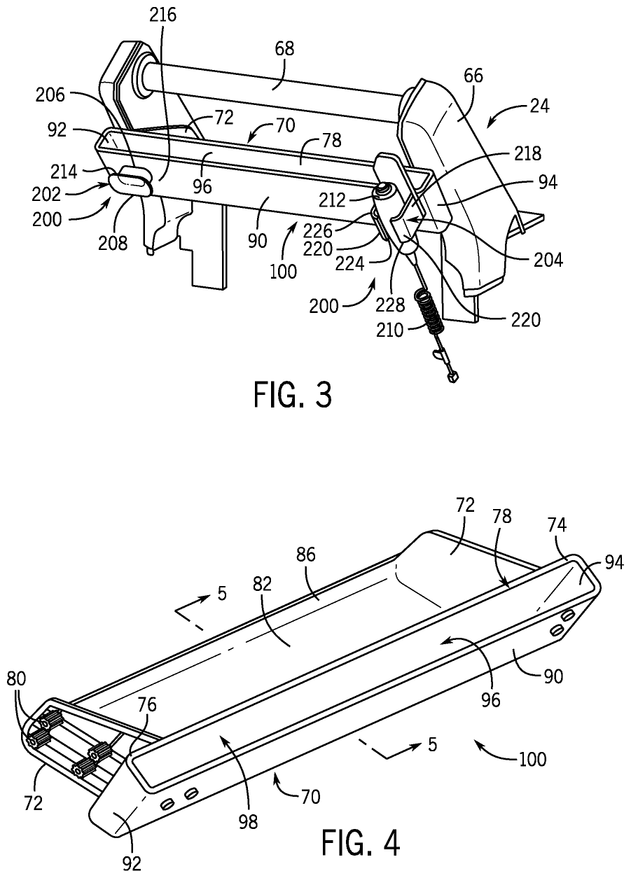

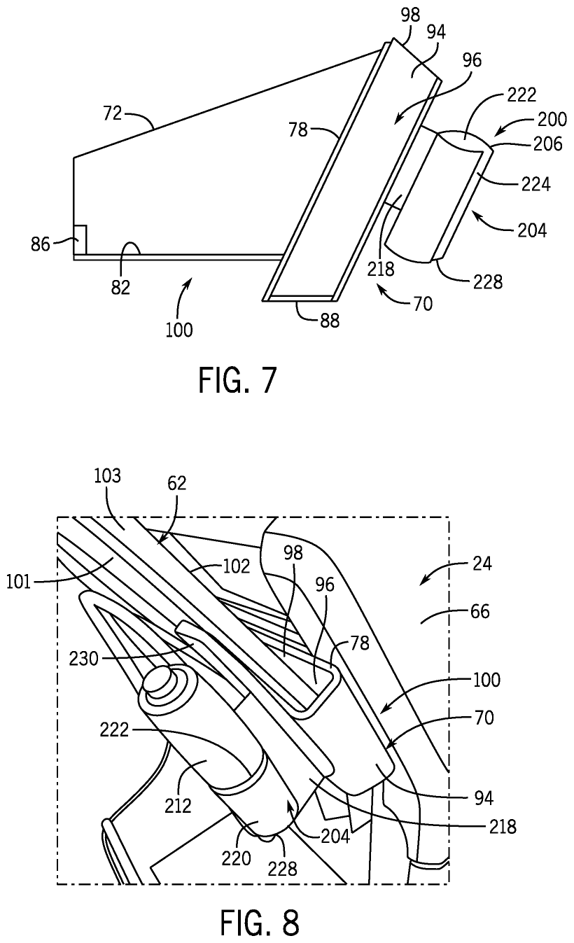

Looking now at the illustrated exemplary embodiment of FIGS. 2-8, the drive handle 24 includes a pair of arms 66 that extend outwardly from the chassis 12 on each side of the body 13. Opposite the body 13, the arms 66 include a bar 68 extending therebetween. The angle of the arms 66 in relation to the body 13 positions the bar 68 at an ergonomically acceptable location where an individual can easily grasp the bar 68 to readily control the movement of the mobile x-ray device 10 while also providing sufficient room for the individual to walk normally behind the mobile x-ray device 10 without inadvertently contacting, e.g., kicking, the mobile x-ray device 10.

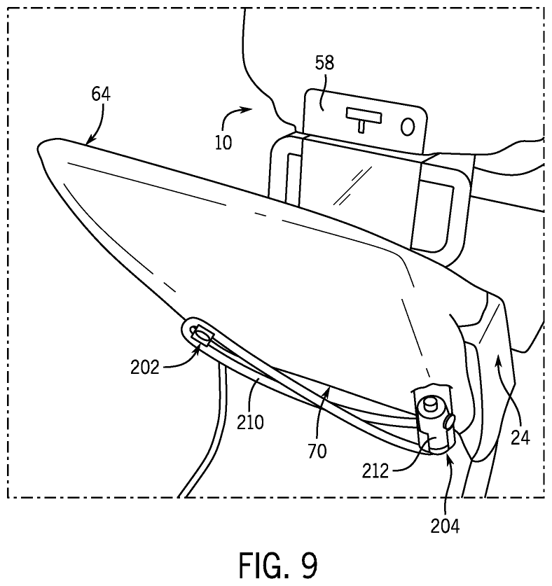

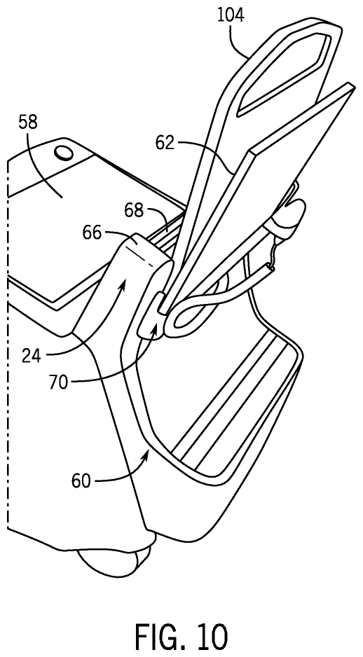

In order to enable the mobile x-ray device 10 to facilitate the placement of a detector 62, e.g., selected from within the bin 60, into a protective bag 64 (FIG. 9) for use in an imaging procedure, the mobile x-ray device 10 includes a detector support 100. The detector support 100 is positioned between the arms 66 of the drive handle 24 and below the bar 68 to allow for easy access to and use of the support 100. As best shown in FIGS. 2-5, the detector support 100 includes a sleeve 70 having a pair of securing plates 72 extending outwardly from positions spaced inwardly from opposite ends 74,76 of rear side wall 78 of the sleeve 70. The securing plates 72 can extend outwardly from the sleeve 70 at any desired angle, but in the illustrated exemplary embodiment the securing plates 72 extend outwardly at a downward angle relative to the sleeve 70. This downward angle enables the securing plates 72 to be affixed to the adjacent arms 66 in a horizontal position, thereby facilitating the engagement of the securing plates 72 with the arms 66, while positioning the sleeve 70 at an angle corresponding to the angle of the arms 66 to prevent the arms 66 from interfering with the insertion of a detector 62 with or without a grid handle 104 (FIG. 10) thereon into the sleeve 70. Additionally, the location of the securing plates 72 spaced inwardly from the ends 74,76 enables the securing plates 72 to be attached to the arms 66 in a configuration that aligns the ends of the sleeve 70 with the outer ends or sides of the arms 66, such that the sleeve 70 does not extend beyond the profile of the drive handle 24. The securing plate 72 can be affixed to the arms 66 in any suitable manner, and in the illustrated exemplary embodiment of FIG. 4 the securing plates 72 include a number of channels 80 formed therein. The channels 80 are aligned with bores (not shown) disposed in the arms 66 and can receive fasteners (not shown) therein to secure the securing plates 72 to the arms 66.

Between the securing plates 72 the detector support 100 can optionally include a support surface 82. The support surface 82 extends between the securing plates 72 and the sleeve 70 to form a shelf located between the arms 66 and forward of the sleeve 70 on which a number of items 84 to be employed by the user/technician during the operation of the mobile x-ray device 10. To prevent items positioned thereon from rolling or otherwise falling off of the support surface 82, the support surface 82 is bounded by the side wall 78 of the sleeve 70, the securing plate 72 and a lip 86 located along the support surface 82 opposite the sleeve 70 and extending between the securing plates 72. Further, the support surface 82 is oriented between the securing plates 72 such that the support surface 82 is disposed in a generally flat, horizontal position when the securing plates 72 are secured to the arms 66. Also, in another exemplary embodiment, the location of the attachment of the securing pates 72 to the arms 66 disposes the support surface 82 at a location relative to the bar 68 where the support surface 82 and any items thereon can be readily accessed either under or over the bar 68.

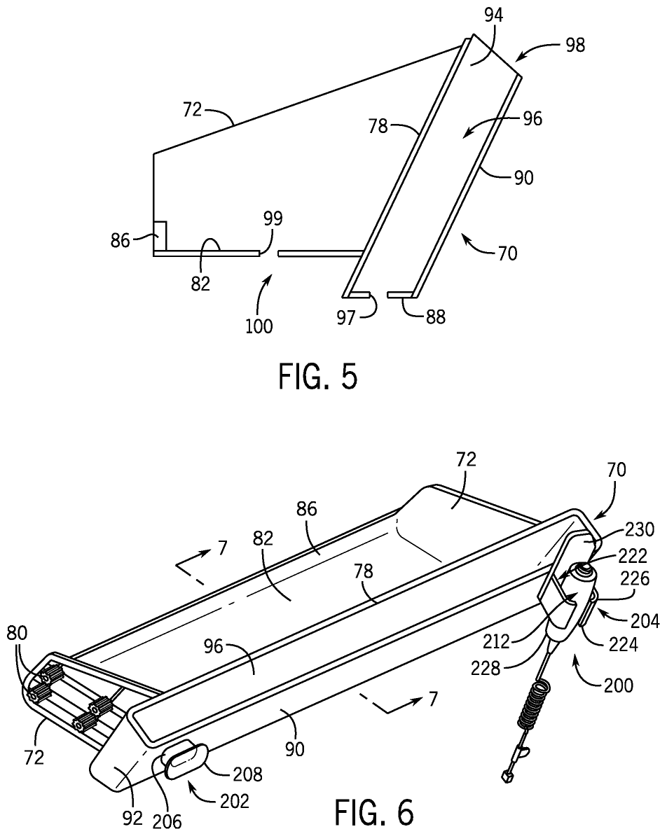

Looking now at the illustrated exemplary embodiment of FIGS. 2-5 and 8-9, the sleeve 70 is disposed along a rear edge of the arms 66, so as not to obscure or interfere with the user console/display 56, and within the boundary or width defined by the outer side edges of the arms 66, to maintain the sleeve 70 within the frame defined by the chassis 12/body 13. The sleeve 70 is formed with a bottom wall 88, the rear side wall 78 that extends upwardly from the bottom wall 88, a front side wall 90 extending upwardly from the bottom wall 88 opposite the rear side wall 78, and a pair of end walls 92,94 extending upwardly from the bottom wall 88 and joining the adjacent edges of the rear side wall 78 and the front side wall 90. The sleeve 70 defines an interior 96 accessible through an open top end 98 that can receive the entirety of one side edge 103 of a detector 62 therein. The space defined by the interior 96 can be selected as desired and is capable of enabling the entire thickness or width and length of one side edge 103 of the detector 62, again with or without the grid handle 104 (FIG. 10), to be placed therein. In the illustrated exemplary embodiment, the bottom wall 88 is formed to be flat, or parallel in orientation to the support surface 82. Further, the open top end 98 is oriented at an angle with regard to the bottom wall 88 and support surface 82, such as to be aligned with the orientation of the arms 66. In the exemplary embodiment best shown in FIGS. 4 and 5, the height of the front side wall 90 from the bottom wall 88 is shorter than the height of the rear side wall 78, with a corresponding slope formed on each of the end walls 92,94 between the rear side wall 78 and the front side wall 90 at the open top end 96. In addition, to facilitate the cleaning of the interior 96, the bottom wall 88 can be formed with one or more apertures 97 therein to allow smaller particulate matter and liquids to pass through the bottom wall 88. The support surface 82 can also include apertures 99 therein for this purpose. In an alternative embodiment, the support surface 82 and/or the bottom wall 88 can be formed with a slope (not shown) to direct any small particulate matter and/or liquids towards the apertures 97,99 disposed at the lowest point of the slope.

As best shown in FIGS. 4-7, the bottom wall 88 and the open top end 98 are spaced from one another a sufficient length that the entire length of each of the rear side wall 78 and front side wall 90 can engage the opposed surfaces 101,102 of the detector 62 with or without a grid handle 104 along their entire length in positions where an entire edge 103 of the detector 62 with or without a grid handle 104 is securely retained within the interior 96 of the sleeve 70. Further, the spacing of the rear side wall 78 and the front side wall 90 by the end walls 92,94 is greater than the thickness of the detector 62, with or without a grid handle 104, such that in the illustrated exemplary embodiment the detector 62 can be pivoted and/or angled within the interior 96 along the edge 103 to an angle away from the rear side wall 78. In this position, the rear side wall 78 and the front side wall 90 can securely engage opposed surfaces 101,102 of the detector 62 with or without a grid handle 104 while positioning enough of the detector 62 with or without a grid handle 104 outside of the sleeve 70 to allow the user/technician to readily position a protective bag 64 around the exposed portion of the detector 62 with or without a grid handle 104 in a hands-free manner, i.e., without needing to manually hold any portion of the detector 62 with or without a grid handle 104. When the open end of the bag 64 has been positioned around the exposed portion of the detector 62 with or without a grid handle 104, as shown in FIG. 9, the detector 62 with or without a grid handle 104 can be removed from the sleeve 70 and inverted in order to insert the closed end of the bag 64 and the detector 62 with or without a grid handle 104 disposed therein into the sleeve 70. In this position, the user/technician can close the open end of the bag 64 around the detector 62 with or without a grid handle 104 in a hands-free manner with the detector 62 with or without a grid handle 104 and bag 64 supported within the sleeve 70 to prepare the detector 62 for use in an imaging procedure. After use in an imaging procedure, this process can be reversed, i.e., placing the detector 62 with or without a grid handle 104 and closed bag 64 in the sleeve 70 to open the bag 64, removing the detector 62 with or without a grid handle 104 and open bag 64 from the sleeve 70, inverting the detector 62 with or without a grid handle 104 and open bag 64, and reinserting the detector 62 with or without a grid handle 104 and open bag 64 into the sleeve 70 to allow for hands-free removal of the bag 64 from the detector 62 with or without a grid handle 104.

Further, the areas of the surfaces 101,102 (FIG. 8) of the detector 62 that are exposed when the detector 62 with or without a grid handle 104 is positioned within the interior 96 of the sleeve 70 allows for easy cleaning and drying of the surfaces 101,102, particularly after use of the detector 62 with or without a grid handle 104 in an imaging procedure and removal of the bag 64, again in hands-free manner, i.e., without having to manually hold any portion of the detector 62 with or without a grid handle 104 during the cleaning and drying process. Also, with regard to FIG. 10, the width of the interior 96 being greater than that of the detector 62 allows for the assembly or disassembly of a grid handle 104 from the detector 62 while the detector 62 and grid handle 104 are securely held within the interior 96 of the detector support 100. In one particular exemplary embodiment of the detector support 100 to facilitate this use of the detector support 100, the front side wall 90 has a height from the bottom wall 88 of from about 2″ to about 5″, and more specifically from about 3″ to about 4″, and most particularly about 3.5″.

Referring now to FIGS. 2-3 and 6-9, the front side wall 90 of the sleeve 70 can include a cord management system 200. The cord management system 200 includes a cord wrap 202 and a switch holder 204. In the illustrated exemplary embodiment, the cord wrap 202 is disposed adjacent side wall 92 and the switch holder 204 is disposed adjacent side wall 94, though other configurations for the cord wrap 202 and/or the switch holder 204 are within the scope of the present disclosure.

The cord wrap 202 is secured to the front side wall 90 opposite the interior 96, such as by an adhesive or mechanical fastener (not shown), e.g., one or more screws, engaged between the cord wrap 202 and the front side wall 90. The cord wrap 202 includes a narrow end 206 disposed adjacent the front side wall 90 and a wide end 208 extending outwardly from the narrow end 206 opposite the front side wall 90. The wide end 208 has an outer periphery dimension that extends at least partially beyond the narrow end 206 to provide an attachment location for the cord 210 connecting the X-ray exposure switch 212 to the body 13. In the illustrated exemplary embodiment, the wide end 208 has an oval shape with a perimeter 214 that defines a channel 216 between the wide end 208 and the front side wall 90. The cord 210 can be disposed around the narrow end 206 between the wide end 208 and the front side wall 90 to maintain the cord 210 in a storage position adjacent the front side wall 90, as shown in FIGS. 2 and 9.

The switch holder 204 is secured to the front side wall 90 opposite the interior 96, such as by an adhesive or mechanical fastener (not shown), e.g., one or more screws, engaged between the switch holder 204 and the front side wall 90. In the illustrated exemplary embodiment, the switch holder 204 includes a base 218 adjacent the front side wall 90 and a pair of retaining arms 220 disposed on and extending outwardly away from opposed sides of the base 218 to define a retaining space 222 therebetween. The retaining arms 220 extend from the base 218 inwardly towards one another and are separated opposite the base 218 by a slot 224 disposed between the retaining arms 220. The retaining space 222 can also optionally be defined by the retaining arms 220 including a taper or narrowing from a wide upper end 226 to a narrow lower end 228. The dimensions of the retaining space 222 defined by the retaining arms 220 enables the activation switch 212 to be placed into the retaining space 222 through the upper end 226 to engage or rest upon the lower end 228, The cord 210 connecting the activation switch 212 to the body 13 can be positioned within the slot 224 to allow the activation switch 212 to be fully positioned within the switch holder 204 without interference of the cord 210.

When using the switch holder 204, to prevent the cord 210 and/or switch 212 from inadvertently being positioned within the interior 96 of the sleeve 70 the switch holder 204 includes a guide 230. The guide 230 extends upwardly from the base 218 above the front side wall 90. Thus, the guide 230 functions to stop the switch 212 from being positioned above the interior 96 of the sleeve 70 when moved towards the switch holder 204 and directs the cord 210 and/or the activation switch 212 into the switch holder 204. Additionally, in an alternative embodiment multiple guide 230 can be disposed on and spaced along the length of the front side wall 90 adjacent the open upper end 92 to provide a similar function to direct the detector 60 into the interior 96 of the sleeve 70.

The detector support 100 including the sleeve 70, and optionally including the support surface 82 and one or both of the cord wrap 202 and switch holder 204 of the cord management system 200, can be formed from any suitable material, such as a suitable metal or plastic material. The detector support 100 disclosed herein can be formed as a unitary member of from different component members secured to one another. Further, the detector support 100 including the sleeve 70 can be utilized as a retrofit component to an existing mobile x-ray device 10, where the sleeve 70 is secured to a drive handle 24 of the mobile x-ray device 10 adjacent a shelf (not shown) already disposed on the drive handle 24, or the shelf directly.

It is understood that the aforementioned compositions, apparatuses and methods of this disclosure are not limited to the particular embodiments and methodology, as these may vary. It is also understood that the terminology used herein is for the purpose of describing particular exemplary embodiments only, and is not intended to limit the scope of the present disclosure which will be limited only by the appended claims.

We claim:

1. A detector support for a mobile x-ray device, the detector support comprising:
a. a sleeve defining an interior adapted to receive an edge of a detector therein; and
b. a pair of securing plates extending outwardly from the sleeve and adapted to be secured to the mobile x-ray device.

2. The detector support of claim 1, wherein the sleeve comprises:
a. a bottom wall;
b. a rear side wall extending upwardly from the bottom wall;
c. a front side wall extending upwardly from the bottom wall opposite the rear side wall;
d. a pair of end walls extending upwardly from the bottom wall and joining opposed ends of the rear side wall and the front side wall, and
e. an open upper end disposed opposite the bottom wall.

3. The detector support of claim 2, wherein the rear side wall and front side wall are disposed at an angle with regard to the bottom wall.

4. The detector support of claim 3, wherein the front side wall is shorter than the rear side wall.

5. The detector support of claim 4, wherein the front side wall has a height between about 2″ and about 5″.

6. The detector support of claim 1, wherein the securing plates extend at a downward angle relative to the rear side wall.

7. The detector support of claim 6, further comprising a support surface disposed between the sleeve and the pair of securing plates.

8. The detector support of claim 1, further comprising a cord management system disposed on the front side wall opposite the interior.

9. The detector support of claim 8, wherein the cord management system comprises
a. a cord wrap disposed on the front side wall; and
b. a switch holder disposed on the front side wall.

10. A mobile x-ray device comprising:
a. a body including a number of wheels rotatably attached thereto and housing operative systems for the obtaining and processing of x-ray data to provide x-ray images;
b. a moveable vertical column and horizontal arm operably connected to the body and including an x-ray emitter opposite the body;
c. a detector configured to receive x-rays from the x-ray emitter and operably connected to the operative systems to transmit x-ray data from the detector to the operative systems;
d. a drive handle disposed on the body; and
e. a detector support comprising:
i. a sleeve defining an interior adapted to receive an edge of a detector therein; and
ii. a pair of securing plates extending outwardly from the sleeve and secured to the drive handle.

11. The mobile x-ray device of claim 10, wherein the sleeve comprises:
a. a bottom wall;
b. a rear side wall extending upwardly from the bottom wall;
c. a front side wall extending upwardly from the bottom wall opposite the rear side wall;
d. a pair of end walls extending upwardly from the bottom wall and joining opposed ends of the rear side wall and the front side wall, and
e. an open upper end disposed opposite the bottom wall.

12. The mobile x-ray device of claim 11, wherein the front side wall is shorter than the rear side wall.

13. The mobile x-ray device of claim 12, wherein the front side wall has a height between about 2″ and about 5″.

14. The mobile x-ray device of claim 10, further comprising a support surface disposed between the sleeve and the pair of securing plates.

15. The mobile x-ray device of claim 10, further comprising a cord management system disposed on the front side wall opposite the interior.

16. The mobile x-ray device of claim 15, wherein the cord management system comprises
a. a cord wrap disposed on the front side wall; and
b. a switch holder disposed on the front side wall.

17. The mobile x-ray device of claim 10, wherein the sleeve does not extend beyond outer sides of the drive handle.

18. A method of supporting a detector on a mobile x-ray device, the method comprising the steps of:
a. providing a mobile x-ray device comprising:
i. a body including a number of wheels rotatably attached thereto and housing operative systems for the obtaining and processing of x-ray data to provide x-ray images;
ii. a moveable vertical column and horizontal arm operably connected to the body and including an x-ray emitter opposite the body;
iii. a detector configured to receive x-rays from the x-ray emitter and operably connected to the operative systems to transmit x-ray data from the detector to the operative systems;
iv. a drive handle disposed on the body; and
v. a detector support comprising:
1. a sleeve defining an interior adapted to receive an edge of a detector therein; and
2. a pair of securing plates extending outwardly from the sleeve and secured to the drive handle; and
b. placing an edge of the detector within the sleeve to support the detector within the sleeve in a hands-free manner; and
c. placing a protective bag around the detector.

19. The method of claim 18, further comprising the steps of:
a. performing an imaging procedure with the detector within the protective bag;
b. placing the detector and protective bag within the sleeve; and
c. removing the protective bag from the detector.

20. The method of claim 18, further comprising cleaning the detector within the sleeve after removing the protective bag.
